(19) United States
(12) Patent Application Publication
(10) Pub. No.: US 2024/0397183 A1
(43) Pub. Date: Nov. 28, 2024

(54) IMAGE PICKUP UNIT AND ENDOSCOPE

(71) Applicant: OLYMPUS MEDICAL SYSTEMS CORP., Tokyo (JP)

(72) Inventors: Masateru SUZUKI, Tokyo (JP); Shinya ISHIKAWA, Tokyo (JP)

(73) Assignee: OLYMPUS MEDICAL SYSTEMS CORP., Tokyo (JP)

(21) Appl. No.: 18/796,848

(22) Filed: Aug. 7, 2024

Related U.S. Application Data
(63) Continuation of application No. PCT/JP2022/022129, filed on May 31, 2022.

(51) Int. Cl. H04N 23/50; A61B 1/05

(52) U.S. Cl. CPC H04N 23/555; A61B 1/051

(57) ABSTRACT

The invention includes: an image pickup device having a light receiving surface and a back surface; a first substrate having a first main surface, a second main surface, and four side surfaces, the first substrate comprising a stacked substrate including internal wirings; and a second substrate having a third main surface, a fourth main surface, and four side surfaces. A solder ball on the back surface and an electrode on the first main surface are bonded an electrode on the second main surface and an electrode on the third main surface are bonded, end portions of the internal wirings are, of the four side surfaces, not exposed on a first side surface and a second side surface, and exposed on a third side surface and a fourth side surface, and the exposed end portions are arranged only in a single layer of the stacked substrate.

### CROSS REFERENCE TO RELATED APPLICATION

This application is a continuation application of PCT/JP2022/022129 filed on May 31, 2022, the entire contents of which are incorporated herein by this reference.

### BACKGROUND OF THE INVENTION

### 1. Field of the Invention

This invention relates to an image pickup unit comprising a plurality of bonded substrates, and an endoscope including the image pickup unit.

### 2. Description of the Related Art

Conventionally, endoscopes, etc. have been widely used in, e.g., the medical field, industrial field, etc. A medical endoscope used in a medical field has a function to insert an insertion portion provided with an image pickup unit, and the like, into a body cavity of a living body, and acquire and display an image of a lesion part, etc. inside an organ, etc. The user can observe and inspect the lesion part, etc. based on the image acquired and displayed by the endoscope.

Conventional image pickup units used for this kind of endoscope, etc. include, e.g, one configured in a form in which an image pickup device and a plurality of wiring board are connected in order from the front end towards the back end, and a cable is connected to a wiring board on the side of the back end.

As an image pickup unit with such a conventional configuration, there have been put into practical use an image pickup unit in which all or a part of a plurality of wiring boards employ so-called a stacked substrate formed by stacking a plurality of substrates each forming an internal wiring. For this kind of image pickup unit that employed a stacked substrate, various proposals such as Japanese Patent No. 6013657, etc. have been made.

The stacked substrate employed in a conventional image pickup unit is generally formed as a collective substrate in the form of, e.g., a plurality of a plurality of substrates collected together. In this case, wiring is formed on the surface layer or inner layer of each stacked substrate. As the wiring material, e.g., copper (Cu) or tungsten (W) is used.

In this kind of stacked substrate, the wiring exposed on the surface layer is generally treated with, e.g., electroplating (Ni—Au) to prevent corrosion, etc. and ensure good solderability. To this end, current-carrying wiring (what is called a plated draw wire) is formed on the collective substrate to ensure current carrying during the plating process. This current-carrying wiring becomes the form of being exposed on the end surface of each stacked substrate after the individualisation (dicing) of each stacked substrate from the collective substrate. Because the exposed surface is formed after the electroplating process, no electroplating is formed on the exposed surface and the wiring material (Cu, W) is in the state of being exposed. In this case, the current-carrying wiring of the stacked substrate is usually in the form of, e.g., suitably disposed on the four side surfaces of each stacked substrate.

### SUMMARY OF THE INVENTION

In order to achieve the above-mentioned objective, an image pickup unit according to one aspect of the invention includes: an image pickup device having a light receiving surface and a back surface on an opposite side to the light receiving surface; a first substrate having a first main surface, a second main surface on an opposite side to the first main surface, and at least four side surfaces covering an area including a periphery of the first main surface and the second main surface, the first substrate comprising a stacked substrate including a plurality of internal wirings; and a second substrate having a third main surface, a fourth main surface on an opposite side to the third main surface, and at least four side surfaces covering an area including a periphery of the third main surface and the fourth main surface. An electrode on the back surface of the image pickup device and an electrode on the first main surface of the first substrate are bonded to each other, an electrode on the second main surface of the first substrate and an electrode on the third main surface of the second substrate are bonded to each other, a plurality of end portions of the internal wirings are, of the four side surfaces of the first substrate, not exposed on a first side surface and a second side surface on an opposite side to the first side surface, and exposed on a third side surface and a fourth side surface orthogonal to the first side surface and the second side surface, and the exposed end portions of the internal wirings are arranged only in a single layer of the stacked substrate of the first substrate.

An image pickup unit according to another aspect of the invention includes: an image pickup device having a light receiving surface and a back surface on an opposite side to the light receiving surface; a first substrate having a first main surface, a second main surface on an opposite side to the first main surface, and at least four side surfaces covering an area including a periphery of the first main surface and the second main surface, the first substrate comprising a stacked substrate including a plurality of internal wirings; and a second substrate having a third main surface, a fourth main surface on an opposite side to the third main surface, and at least four side surfaces covering an area including a periphery of the third main surface and the fourth main surface. An electrode on the back surface of the image pickup device and an electrode on the first main surface of the first substrate are bonded to each other, an electrode on the second main surface of the first substrate and an electrode on the third main surface of the second substrate are bonded to each other, a plurality of end portions of the internal wirings are, of the four side surfaces of the first substrate, not exposed on a first side surface and a second side surface on an opposite side to the first side surface, and exposed on a third side surface and a fourth side surface orthogonal to the first side surface and the second side surface, and the plurality of exposed end portions of the internal wirings are arranged at positions that do not overlap with each other in a stacking direction.

An endoscope according to one aspect of the invention includes an insertion portion to be inserted into a subject and an image pickup unit provided at a distal end of the insertion unit. The image pickup unit includes: an image pickup device having a light receiving surface and a back surface on an opposite side to the light receiving surface; a first substrate having a first main surface, a second main surface on an opposite side to the first main surface, and at least four side surfaces covering an area including a periphery of the first main surface and the second main surface, the first substrate comprising a stacked substrate including a plurality of internal wirings; and a second substrate having a third main surface, a fourth main surface on an opposite side to the third main surface, and at least four side surfaces covering an area including a periphery of the third main surface and the fourth main surface. An electrode on the back surface of the image pickup device and an electrode on the first main surface of the first substrate are bonded to each other, an electrode on the second main surface of the first substrate and an electrode on the third main surface of the second substrate are bonded to each other, a plurality of end portions of the internal wirings are, of the four side surfaces of the first substrate, not exposed on a first side surface and a second side surface on an opposite side to the first side surface, and exposed on a third side surface and a fourth side surface orthogonal to the first side surface and the second side surface, and the exposed end portions of the internal wirings are arranged only in a single layer of the stacked substrate of the first substrate.

### BRIEF DESCRIPTION OF THE DRAWINGS

FIG. 1 is an external perspective view showing the schematic configuration of an image pickup unit of a first embodiment of the invention.

FIG. 2 is an external perspective view taking out and showing only a first substrate of the image pickup unit in FIG. 1.

FIG. 3 is an external perspective view showing the schematic configuration of an internal wiring included in the first substrate of the image pickup unit in FIG. 1.

FIG. 4 is a side view seen from the direction shown with the arrow reference sign [4] in FIG. 2.

FIG. 5 is an external perspective view taking out and showing only a second substrate of the image pickup unit in FIG. 1.

FIG. 6 conceptually shows the situation of the cable bonding process in the manufacturing process of the image pickup unit in FIG. 1.

FIG. 7 is an external perspective view showing a modification of a first substrate in the image pickup unit of FIG. 1.

FIG. 8 is an external perspective view showing the schematic configuration of an image pickup unit of a second embodiment of the invention

FIG. 9 is an external perspective view taking out and showing only a first substrate of the image pickup unit in FIG. 8.

FIG. 10 is an external perspective view showing the schematic configuration of an internal wiring included in the first substrate of the image pickup unit in FIG. 8.

FIG. 11 is a side view seen from the direction shown with the arrow reference sign [11] in FIG. 9.

FIG. 12 is an exterior view showing the schematic configuration of an endoscope system including the endoscope employing the image pickup unit of either FIG. 1 or 8.

### DETAILED DESCRIPTION OF THE PREFERRED EMBODIMENTS

In general, image pickup units used for endoscopes, etc. always have the request of miniaturization. To this end, in recent years, the image pickup units used for endoscopes, etc. have become highly miniaturized parts whose image pickup surface has, e.g., vertical and horizontal sizes about several millimeters.

In the conventional image pickup unit as such a miniaturized part, at the time of manufacturing, there is an operation process which solder-bond a cable, etc. to, e.g., the wiring board on the back end side, or an operation process which bonds a distal end glass, etc. on the front surface of an image pickup device. At the time of these operation processes, the image pickup unit may be handled by the worker using a tool such as a tweezer. Specifically, the external surface of the image pickup unit may be grasped using, e.g., this tool. As tools used in this type of operation process, tools with electrical conductivity are generally used to restrain the target part (image pickup unit) from being charged.

Here, in order to hold the image pickup unit stably using a predetermined tool, as flat a part of the outer surface of the image pickup unit as possible will be grasped by the tool. However, since miniaturization of image pickup units is progressing in recent years as described above, the part which is easy to grasp by a tool is limited. At the part (superficial part) which is easy to grasp by a tool, the end surface of the above-mentioned current-carrying wiring may be formed exposed.

Therefore, in the manufacturing process of an image pickup unit, etc., when grasping the image pickup unit using an electrically conductive tool, such as a tweezer, the end surface of the current-carrying wiring exposed to the side surface of a stacked substrate and the tool may have contacted to each other. This may result in the current-carrying wiring being short-circuited by the tool. In such a case, damage may be caused to the image pickup device mounted in the image pickup unit.

On the other hand, the current-carrying wiring may be formed in a plurality of layers in the stacked substrate applied to the conventional image pickup unit. At this time, the wirings for current carrying provided in respective substrates in the stacked substrate may be arranged to oppose to each other in the stacking direction. In the stacked substrate of such configuration, an electric field will produce among the current-carrying wiring when the product is energized at the time of use. This electric field is known to be significant in the field in which the wirings for current carrying are arranged to oppose to each other.

The conventional image pickup unit may be used in a high humidity situation, e.g., in a case of being used for an endoscope, etc.

As mentioned above, in the stacked substrate in the image pickup unit, the end surface of the wiring material (such as Cu) of the current-carrying wiring is exposed on either side surface of the substrate. Therefore, corrosion may occur at the exposed part of the current-carrying wiring. This may cause a situation where metallic ions of the wiring material are eluted from the part.

In this case, the eluted metallic ions may deposit around the current-carrying wiring in the vicinity of the electric field produced between the current-carrying wirings. This causes a concern that the deposited metallic ions may short-circuit the current-carrying wiring.

According to the present invention, it is enabled to provide an image pickup unit and an endoscope including the image pickup unit, in which the image pickup unit employs a stacked substrate that is capable of restraining short circuits that may occur due to the current-carrying wiring of the stacked substrate during product assembly or product use.

Hereinafter, the invention will be described by means of the embodiments shown in the figures. The drawings used in the following descriptions are schematic and may show the dimensional relationship and scale of each component, etc. differently for each component, in order to show each component at a size large enough to be recognized on the drawing. Therefore, the invention is not limited only to the illustrated form with regard to the quantity of each component, the shape of each component, the ratio of the size of each component, and the relative position of each component, etc. described in each drawing.

### First Embodiment

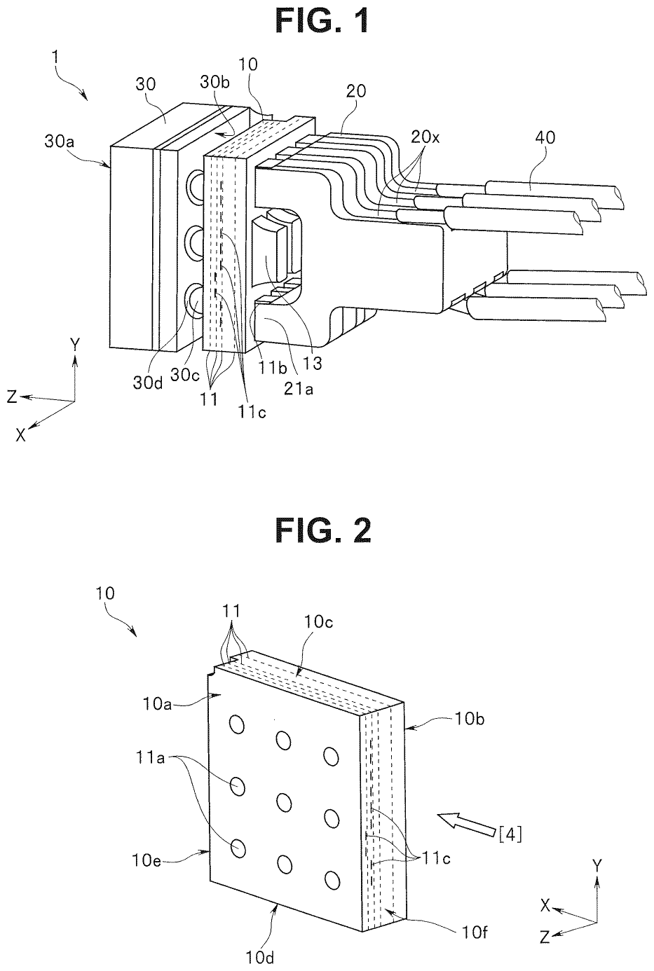

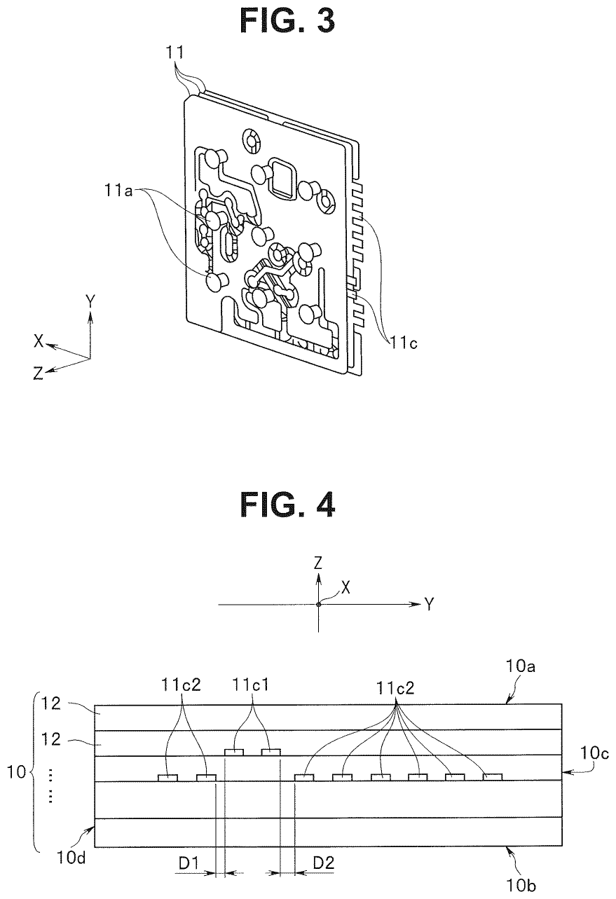

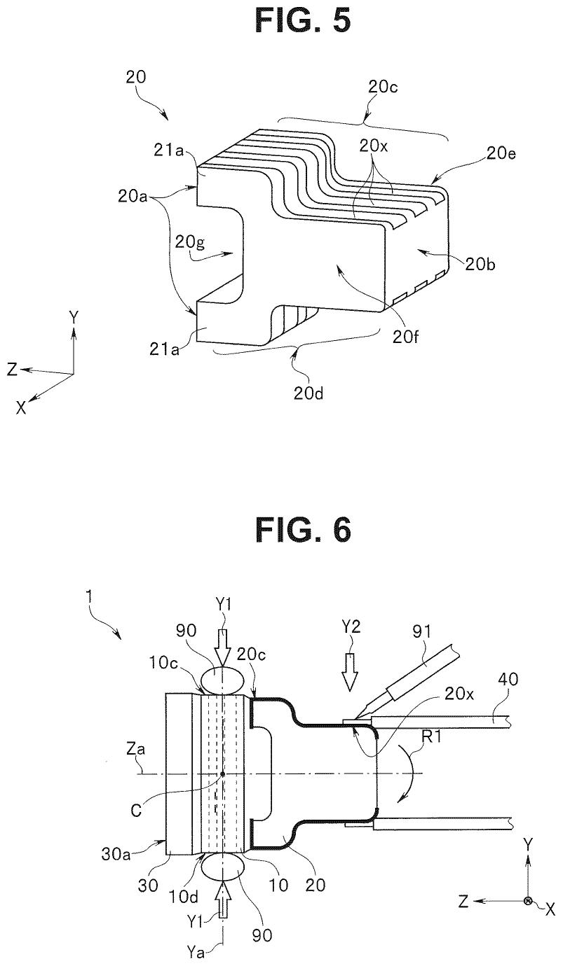

FIGS. 1 to 5 show an image pickup unit of a first embodiment of the invention. Of these, FIG. 1 is an external perspective view showing the schematic configuration of an image pickup unit of a first embodiment of the invention. FIG. 2 is an external perspective view taking out and showing only a first substrate of the image pickup unit in FIG. 1. FIG. 3 is an external perspective view showing the schematic configuration of an internal wiring included in the first substrate of the image pickup unit in FIG. 1. FIG. 4 is a side view seen from the direction shown with the arrow reference sign [4] in FIG. 2. FIG. 5 is an external perspective view taking out and showing only a second substrate of the image pickup unit in FIG. 1.

It is noted that arrows X and Y shown in FIGS. 1 to 5 show the plane of a light receiving surface 30a of an image pickup device 30. That is, arrow X shows the horizontal axis of the light receiving surface 30a, and arrow Y shows the vertical axis of the light receiving surface 30a. Here, arrows X and Y show axes orthogonal to each other. Arrow Z in FIGS. 1 to 5 shows an axis orthogonal to the light receiving surface 30a.

As shown in FIG. 1, the image pickup unit 1 includes a first substrate 10, a second substrate 20, an image pickup device 30, and a plurality of connection cables 40.

The image pickup device 30 is an electronic part composed of a wiring board, etc. which mounted an electronic circuit including a photoelectric conversion device which receives an optical image of a subject (observation object) formed with an optical lens not shown, converts the received image to image data, and outputs the image data. The image pickup device 30 has the light receiving surface 30a and a back surface 30b on an opposite side to the light receiving surface 30a. The light receiving surface 30a of the image pickup device 30 has substantially square shape when seen from the front.

A plurality of lands 30d are provided on the back surface 30b of the image pickup device 30, and a solder ball 30c is provided on each of the lands 30d. The plurality of solder balls 30c are bonded to an electrode 11a provided on the first substrate 10 mentioned below. Thereby, the image pickup device 30 and the first substrate 10 are electrically connected to each other.

It is assumed that the image pickup device 30 employed in the image pickup unit 1 of this embodiment employs an image pickup device similar to one generally applied in conventional endoscopes, etc. The detailed explanation thereof is therefore omitted.

The first substrate 10 is a stacked substrate formed by stacking a plurality of internal wirings 11 and a plurality of base substrates 12 (see, e.g., FIG. 4). For each of the internal wirings 11, e.g. copper (Cu) or tungsten (W) is used. For the plurality of base substrates 12, e.g. a ceramic substrate made of alumina, etc. or a glass epoxy substrate is used.

As shown in FIG. 2, the first substrate 10 has a first main surface 10a, a second main surface 10b on an opposite side to the first main surface 10a, and at least four side surfaces (10c, 10d, 10e, 10f) covering an area including the periphery of the first main surface 10a and the second main surface 10b, and is formed in an overall substantially rectangular shape. Here, the four side surfaces refer to a first side surface 10c, a second side surface 10d, a third side surface 10e, and a fourth side surface 10f, respectively.

In this case, the first main face 10a and the second main face 10b are arranged parallel to each other. The first main faces 10a and the second main face 10b are substantially parallel also to the light receiving surface 30a of the image pickup device 30. Each of the four side surfaces 10c, 10d, 10e, 10f is arranged substantially orthogonal to the first main surface 10a and the second main surface 10b.

The first side surface 10c and the second side surface 10d are substantially parallel to each other. A surface on the opposite side to the first side surface 10c is the second side surface 10d. The third side surface 10e and the fourth side surface 10f are surfaces orthogonal to the first side surface 10c and the second side surface 10d. At this time, the third side surface 10e and the fourth side surface 10f are substantially parallel to each other. A surface on the opposite side to the third side surface 10e is the fourth side surface 10f.

A plurality of electrodes 11a are provided on the first main surface 10a of the first substrate 10 and a plurality of electrodes 11b (not shown in FIG. 2; see FIG. 1) are provided on the second main surface 10b. The plurality of electrodes 11a and 11b are coated with plating, specifically, e.g., processed with electroplated (Ni—Au).

The electrodes 11a on the first main surface 10a of the first substrate 10 are bonded to a plurality of solder balls 30c on the back surface 30b of the image pickup device 30. Thus, the two (the first substrate 10 and the image pickup device 30) are electrically connected to each other.

Similarly, the plurality of electrodes 11b on the second main surface 10b of the first substrate 10 are bonded to a plurality of electrodes 21a (see FIGS. 1 and 5) on a third main surface 20a (see FIG. 5) of the second substrate 20 to be described later. Thus, the first substrate 10 and the second substrate 20 are electrically connected to each other.

In the image pickup unit 1 of this embodiment, the first substrate 10 employs a stacked substrate as described above. The first substrate 10 as a stacked substrate is formed, e.g., as a collective substrate in the form of a plurality of substrates collected together. Here, in the collective substrate, there are formed wiring for energization (so-called plated lead wire) formed to ensure energization when performing plating process on the exposed portions of the internal wiring. The end portion of the current-carrying wiring is exposed on the end surface of each stacked substrate when each stacked substrate is individualized (diced) from the collective substrate.

In the first substrate 10 in the image pickup unit 1 of this embodiment, a plurality of end portions 11c of the current-carrying wiring (internal wiring 11) are configured not to be exposed on the first side surface 10c and the second side surface 10d of the four side surfaces (10c, 10d, 10e, 10f) of the first substrate 10.

In other words, in the first substrate 10, the plurality of end portions 11c of the internal wiring 11 are configured to be exposed on the third side surface 10e and the fourth side surface 10f (see FIGS. 1 and 2). Note that FIGS. 1 and 2 show only the end portions 11c exposed on the fourth side surface 10f. FIGS. 1 and 2 do not show the third side surface 10e itself.

In this case, the plurality of exposed end portions 11c of the internal wiring 11 are arranged side by side in a direction (direction along the arrow Y) orthogonal to the stacking direction (direction along the arrow Z), as shown in FIG. 4.

The plurality of exposed end portions 11c of the internal wiring 11 are arranged at positions that do not overlap with each other in the stacking direction (direction along arrow Z). In other words, as shown in FIG. 4, exposed end portions 11c1 formed on a given substrate layer and exposed end portions 11c2 formed on another substrate layer are arranged at positions that do not overlap in the stacking direction (Z direction). In other words, the exposed end portions 11c1 and the exposed end portions 11c2 are arranged at positions separated by predetermined distance intervals D1, D2 in the direction orthogonal to the stacking direction (Y direction).

As shown in FIG. 1, a plurality of electronic parts 13, etc. are mounted on the second main surface 10b of the first substrate 10.

The second substrate 20 is a Molded Interconnect Device (MID), which is a resin molded product with wirings and electrodes formed. As shown in FIG. 5, the second substrate 20 has a third main surface 20a, a fourth main surface 20b on the opposite side to the third main surface 20a, and at least four side surfaces (20c, 20d, 20e, 20f) covering an area including the periphery of the third main surface 20a and the fourth main surface 20b, and is formed as an overall three-dimensional object with a specified shape. Here, the four side surfaces refer to a fifth side surface 20c, a sixth side surface 20d, a seventh side surface 20e, and an eighth side surface 20f, respectively.

In this case, the third and fourth principal planes 20a and 20b are parallel to each other. The third main faces 20a and the fourth main face 20b are substantially parallel also to the light receiving surface 30a of the image pickup device 30. Each of the four side surfaces 10c, 10d, 10e, 10f is arranged substantially orthogonal to the third main surface 20a and the fourth main surface 20b.

The fifth side surface 20c and the sixth side surface 20d are substantially parallel to each other. A surface on the opposite side to the fifth side surface 20c is the sixth side surface 20d. The fifth side surface 20c and the sixth side surface 20d are parallel to the first side surface 10c and the second side surface 10d of the first substrate 10. The seventh side surface 20e and the eighth side surface 20f are surfaces orthogonal to the fifth side surface 20c and the sixth side surface 20d. At this time, the seventh side surface 20e and the eighth side surface 20f are substantially parallel to each other. A surface on the opposite side to the seventh side surface 20e is the eighth side surface 20f.

The plurality of electrodes 21a are provided on the third main surface 20a of the second substrate 20. The plurality of electrodes 21a are plated (e.g., processed with electroplating (Ni—Au)) in the same manner as the electrodes 11a, 11b of the first substrate 10.

As described above, the plurality of electrodes 11b on the second main surface 10b of the first substrate 10 are bonded to the plurality of electrodes 21a on the third main surface 20a of the second substrate 20 (see FIG. 1). Thus, the first substrate 10 and the second substrate 20 are electrically connected to each other.

At least one of the fifth side surface 20c and the sixth side surface 20d of the second substrate 20 is provided with a bonding electrode 20x to which the connection cables 40 is to be bonded.

In the second substrate 20 illustrated in this embodiment, the third main surface 20a has a recess 20g formed to avoid interference with the electronic parts 13 mounted on the second main surface 10b of the first substrate 10 (see FIG. 5).

The plurality of connection cables 40 are connected to the bonding electrode 20x of the second substrate 20, as described above. The plurality of connection cables 40 are signal transmission cables that transmit output signals, etc. from the image pickup unit 1 to an control apparatus not shown. The plurality of connection cables 40 are signal transmission cables that transmit control signals, etc. output from the control apparatus to the image pickup unit 1.

In the image pickup unit 1 of this embodiment thus configured, the manufacturing process includes, e.g., a operation process of soldering the connection cables 40, etc. to the bonding electrode 20x of the second substrate 20. During this cable bonding process, the image pickup unit 1 is handled using an electrically conductive tool such as a tweezer (hereinafter simply referred to as tool). FIG. 6 conceptually shows the situation of the cable bonding process in the manufacturing process of the image pickup unit of this embodiment.

As shown in FIG. 6, in the process of bonding the connection cable 40 to the image pickup unit 1, etc., the image pickup unit 1 is handled using a tool 90. At this time, the tool 90 may be in the form of grasping two mutually opposed surfaces of the outer surfaces of the image pickup device unit 1.

For example, in the example shown in FIG. 6, the tool 90 is grasping the first side surface 10c and the second side surface 10d (two opposite side surfaces) of the first substrate 10 of the image pickup unit 1. At this time, the grasping force of the tool 90 is applied to each of the first side surface 10c and the second side surface 10d in the direction indicated by arrows Y1 in FIG. 6.

With the image pickup unit 1 thus grasped and secured by the tool 90, the connection cable 40 is soldered to the bonding electrode 20x on the fifth side surface 20c of the second substrate 20. In FIG. 6, a sign 91 indicates a soldering iron. At this time, a force in the direction of an arrow sign Y2 in FIG. 6 is applied to the image pickup unit 1 by the soldering iron 91. As a result, the image pickup unit 1 is subjected to a rotational force (in the direction of arrow R1 in FIG. 6) around the X axis centered at an intersection C between the direction in which the grasping force is applied by the tool 90 (line Ya connecting arrows Y1 in FIG. 6) and a line Za passing through the center of the light receiving surface 30a of the image pickup device 30.

However, at this time, the first side surface 10c and the second side surface 10d are grasped by the tool 90. The grasping force of the tool 90 is holding the image pickup unit 1 against the force in the direction of the arrow R1. Therefore, the image pickup unit 1 is securely secured and held even when subjected to the force in the direction of the arrow R1. Thus, the connection cable 40 is enabled to be securely soldered to the bonding electrode 20x by the soldering iron 91.

As mentioned above, in the first substrate 10 of the image pickup unit 1 of this embodiment, the exposed end portions 11c of the internal wiring 11 are not exposed on the first side surface 10c and the second side surface 10d. In other words, the end portions 11c of the internal wiring 11 are exposed only on the third side surface 10e and fourth side surface 10f. Therefore, the exposed end portions 11c are not short-circuited by the tool 90 even if the first side surface 10c and the second side surface 10d are grasped using a tool 90.

Next, a case is considered of grasping other surfaces of the first substrate 10 and the second substrate 20. For example, the light receiving surface 30a of the image pickup device 30 is a surface that should avoid contact by the tool 90 in consideration of damage, etc. Therefore, the light receiving surface 30a of the image pickup device 30 is not grasped.

The surfaces on the second substrate 20 on which the bonding electrode 20x is provided (the fifth side surface 20c and the sixth side surface 20d) are surfaces on which the bonding operation (soldering operation) of the connection cable 40 is performed. Considering this, the surfaces 20c, 20d should avoid contact by the tool 90 since grasping these surfaces will interfere with the solder bonding operation. For this reason, the fifth side surface 20c and the sixth side surface 20d of the second substrate 20 are also not grasped.

On the other hand, in the first substrate 10, the third side surface 10e and the fourth side surface 10f are smooth surfaces and easy to grasp by a tool. Similarly, in the second substrate 20, the seventh side surface 20e and the eighth side surface 20f are also smooth surfaces and easy to grasp by a tool.

In this case, since there is no internal wiring in the second substrate 20, the cable bonding operation is able to be performed by grasping the seventh side surface 20e and the eighth side surface 20f.

On the other hand, in the configuration of the first embodiment described above, if the third side surface 10e and the fourth side surface 10f of the first substrate 10 are grasped by a tool 90, the exposed end portions 11c of the internal wiring 11 may be short-circuited.

Therefore, in order to grasp the third side surface 10e and the fourth side surface 10f of the first substrate 10 to perform the bonding operation of the connection cable 40, an example such as the following configuration can be considered as another configuration example from the configuration example of the first embodiment described above.

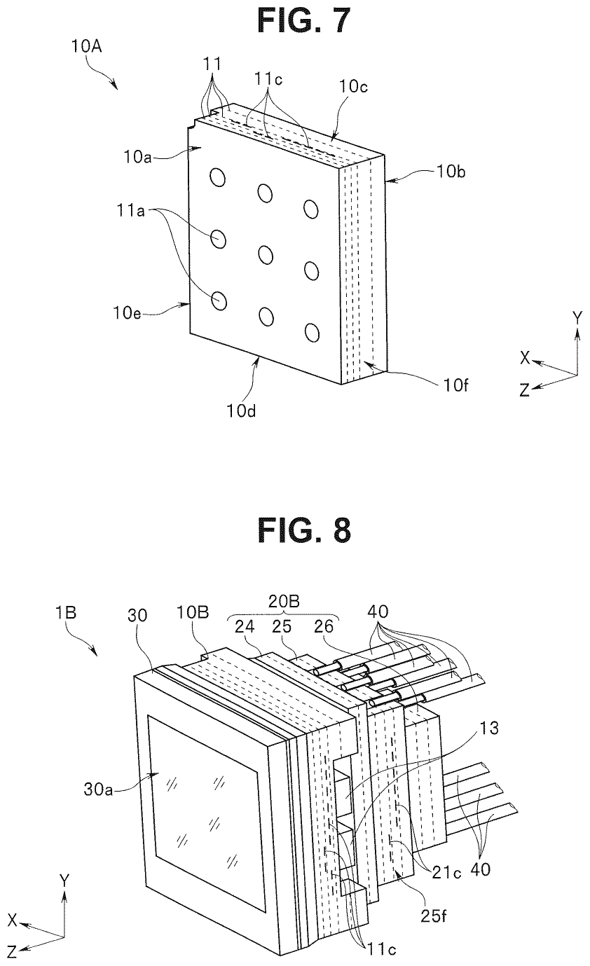

For example, in the modification shown in FIG. 7, the exposed end portions 11c of the internal wiring 11 are not exposed on the third side surface 10e and the fourth side surface 10f of the first substrate 10A. Consequently, the end portions 11c of the internal wiring 11 are exposed on the first side surface 10c and the second side surface 10d.

With this configuration, even if the third side surface 10e and the fourth side surface 10f of the first substrate 10 are grasped using a tool 90, the exposed end portions 11c are not be short-circuited. Therefore, it is enabled to perform the bonding operation of the connection cable 40 while grasping the third side surface 10e and the fourth side surface 10f.

In the case of the configuration shown in the above modification, as in the first embodiment described above, the plurality of exposed end portions 11c of the internal wiring 11 are arranged in a direction (direction along the arrow X in this modification) orthogonal to the stacking direction (direction along the arrow Z).

The plurality of exposed end portions 11c of the internal wiring 11 are arranged at a plurality of positions that do not overlap with each other in the stacking direction (direction along the arrow Z). In other words, as shown in FIG. 7, the exposed end portions 11c formed on a given substrate layer and the exposed end portions 11c formed on another substrate layer are arranged at positions that do not overlap in the stacking direction (Z direction). In other words, the exposed end portions 11c and the exposed end portions 11c are arranged at predetermined intervals in the direction orthogonal to the stacking direction (X direction in this modification), as in the first embodiment above (see also FIG. 4).

As described above, according to the first embodiment, in the image pickup unit 1 connected in the order of the image pickup device 30, the first substrate 10, and the second substrate 20, the first substrate 10 comprises a stacked substrate including the internal wiring 11. In the first substrate 10, the end portions 11c of the internal wiring 11 are configured not to be exposed on the first side surface 10c and the second side surface 10d. That is, in the first substrate 10, the end portions 11c of the internal wiring 11 are configured to be exposed only on the third side surface 10e and the fourth side surface 10f, which are orthogonal to the first side surface 10c and the second side surface 10d.

Thus, the first substrate 10, which is a stacked substrate, is configured such that, of the four side surfaces 10c, 10d, 10e, and 10f, which are easily grasped by the electrically conductive tool 90, the plurality of given end portions 11c of the internal wiring 11 are not exposed on the two predetermined opposite side surfaces (the first side surface 10c and the second side surface 10d).

By using this configuration, when grasping the image pickup unit 1 by a tool 90 during the manufacturing process of the image pickup unit 1, the short circuit of the exposed end portions 11c by the tool 90 can be prevented by grasping the surface on which the plurality of end portions 11c of the internal wiring 11 are not exposed.

A plurality of exposed end portions 11c of the internal wiring 11 are arranged in a direction (X or Y direction) orthogonal to the stacking direction (Z direction). The plurality of exposed end portions 11c of the internal wiring 11 are arranged at a plurality of positions that do not overlap with each other in the stacking direction (Z direction).

Thus, by devising the arrangement of the exposed end portions 11c, it is enabled to make it difficult to generate an electric field in the stacking direction (Z direction) of the exposed end portions 11c that has not been plated, when driving the image pickup unit 1. This can suppress the generation of metallic ions, etc. caused by corrosion, etc. of the exposed end portions 11c. It is thus allowed to prevent short circuits that may occur due to the deposition of metallic ions, etc.

The first substrate 10 of the image pickup unit 1 in this embodiment is illustrated in a configuration in which the exposed end portions 11c of the internal wiring are arranged in a plurality of substrate layers of a stacked substrate. However, there is no limitation on this configuration example. For example, the first substrate 10 may take such a configuration that the end portions 11c of the internal wiring 11 are provided only in a single substrate layer of the plurality of substrate layers.

With such configuration, the end portions 11c of the internal wiring 11 are not arranged overlapped in the stacking direction. Therefore, when driving the image pickup unit 1, the generation of an electric field can be suppressed because there is no electrodes opposed in the stacking direction (Z direction) of the plurality of exposed end portions 11c that have not been plating processed. This can suppress short circuits caused by the deposition of the metallic ions, etc.

The image pickup unit of the first embodiment described above, is shown in a configuration example in which the first substrate 10 is configured of a stacked substrate and a Molded Interconnect Device (MID) is used as the second substrate 20. However, the image pickup unit of the present invention is also able to be configured, e.g., by using a stacked substrate for both the first substrate and the second substrate. The second embodiment of the invention described next is an example of such a configuration.

### Second Embodiment

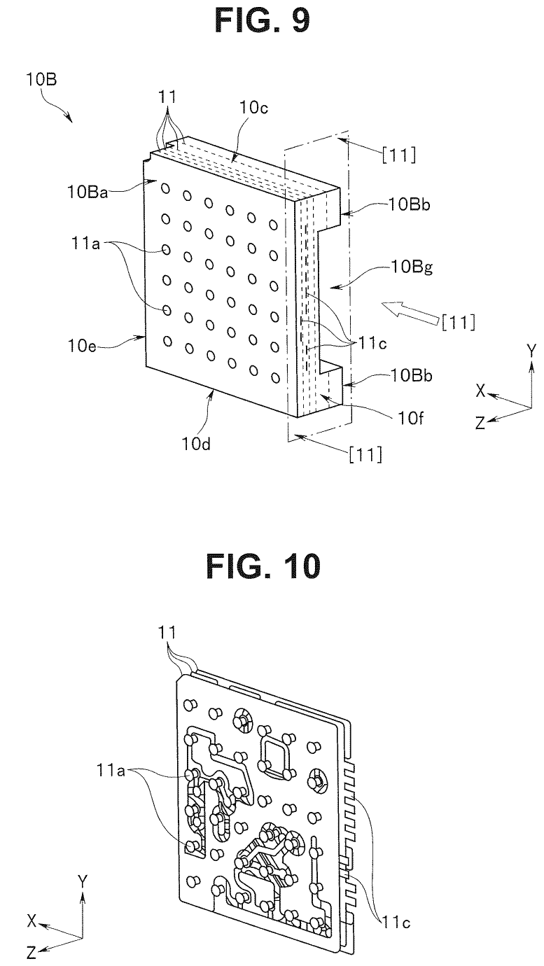

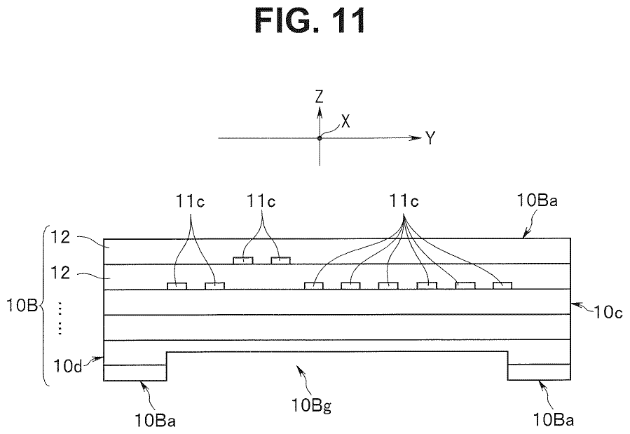

FIGS. 8 to 11 show an image pickup unit of a second embodiment of the invention. Of these, FIG. 8 is an external perspective view showing the schematic configuration of an image pickup unit of a second embodiment of the invention. FIG. 9 is an external perspective view taking out and showing only a first substrate of the image pickup unit in FIG. 8. FIG. 10 is an external perspective view showing the schematic configuration of an internal wiring included in the first substrate of the image pickup unit in FIG. 8. FIG. 11 is a side view seen from the direction shown with the arrow reference sign [11] in FIG. 9.

The basic configuration of this embodiment is substantially similar to that of the first embodiment described above. Therefore, in the following description, the same configuration as that in the first embodiment is indicated with the same symbol and the detailed description thereof is omitted, and only parts differing from the first embodiment described above are described in detail below.

As described above, the configuration of a second substrate 20B of the image pickup unit 1B of this embodiment differs from that of the first embodiment described above.

The second substrate 20B of the image pickup unit 1B of this first embodiment is configured of a plurality of stacked substrates (24, 25, 26) stacked and connected in the Z direction. Here, the stacked substrates are respectively referred to as a first wiring board 24, a second wiring board 25, and a third wiring board 26. The stacked substrates (24, 25, 26) are each configured with a pair of main surfaces, each of which is parallel to the light receiving surface 30a.

The each main surface is provided with a plated electrode (not shown). The electrode on the front main surface of the first wiring board 24 is bonded to the electrode on the back surface of the image pickup device 30. The electrode on the back surface of the first wiring board 24 is bonded to the electrode on the front main surface of the second wiring board 25. The electrode on the back surface of the second wiring board 25 is bonded to the electrode on the front main surface of the third wiring board 26.

The stacked substrates (24, 25, 26) are each configured with four side surfaces, each of which is orthogonal to the light receiving surface 30a. One of the stacked substrates (24, 25, 26) of the second substrate 20B is configured such that a plurality of end portions 21c of the internal wiring are not exposed except on two opposite predetermined side surfaces of the four side surfaces.

In the configuration example shown in FIG. 8, the plurality of end portions 21c of the internal wiring are exposed only on one side surface 25f (corresponding to an eighth side surface) of the second wiring board 25 and on the other side surface (not shown; corresponding to a seventh side surface) on the opposite side surface of the one side surface 25f.

In this case, the plurality of exposed end portions 21c are arranged in a direction (Y direction) orthogonal to the stacking direction (Z direction). The exposed end portions 21c are arranged at positions that do not overlap with each other in the stacking direction (Z direction).

The two predetermined opposite surfaces (corresponding to the fifth and sixth side surfaces) of the four side surfaces of the second wiring board 25 are provided with a bonding electrode to which the the connection cable 40 is to be bonded.

On the other hand, the first substrate 10B is basically the same as the above-described first embodiment in being formed of a stacked substrate. However, the shape of the first substrate 10B in this embodiment is slightly different. For example, as shown in FIG. 9, in a second main surface 10Bb on the opposite side to a first main surface 10Ba, there is formed a recess 10B to avoid interference of the electronic parts 13 with another part (a second substrate 20B) when electronic parts 13 are mounted. Other configurations are substantially similar to the first embodiment described above.

As described above, according to the second embodiment described above, and as in the first embodiment described above, in the first substrate 10B, the end portions 11c of the internal wiring 11 are configured not to be exposed on the first side surface 10c and the second side surface 10d. Thus, it is enabled to grasp the first side surface 10c and the second side surface 10d of the first substrate 10B during the bonding operation of the connecting cable 40.

In this embodiment, the second substrate 20B is configured as a stacked substrate. In the second substrate 20B, the end portions 21c of the internal wiring are configured to be exposed on a surface (e.g., the one side surface 25f of the second wiring board 25; see FIG. 8) other than the surface on which the bonding electrode of the connection cable 40 is provided, of the four side surfaces of the second wiring board 25. The end portions 11c of the internal wiring 11 are configured not to be exposed on the four side surfaces of the first wiring board 24.

Therefore, in the case of this configuration, it is allowed to perform a predetermined operation even when the four side surfaces of the second substrate 20B are grasped.

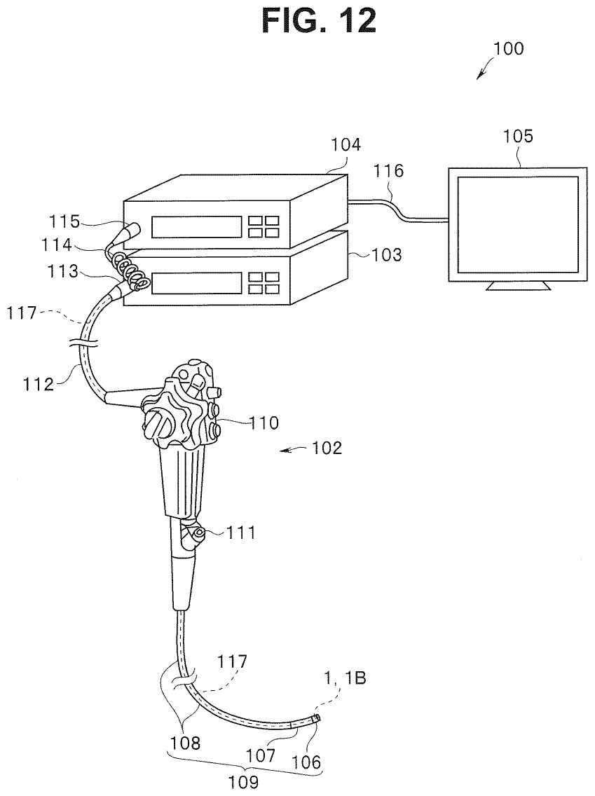

By the way, the image pickup unit (1, 1B) of the above-described each embodiment is employable in an endoscope. Accordingly, the schematic configuration of an endoscope system including an endoscope that employs the image pickup unit of the above-described each embodiment is briefly described below. FIG. 12 is an exterior view showing the schematic configuration of an endoscope system including the endoscope employing the image pickup unit of the above-described each embodiment.

The basic configuration of the endoscope system is substantially similar to that of conventional endoscope systems. Therefore, the following description is limited only to a brief description of each component member of the endoscope system.

As shown in FIG. 12, an endoscope system 100 including an endoscope 102 employing the image pickup unit (1, 1B) of each embodiment of the invention mainly includes the endoscope 102, a light source apparatus 103, a video processor 104, a display apparatus 105, etc.

The endoscope 102 mainly includes an insertion portion 109 having a substantially elongated tubular shape, an operation portion 110 from which the insertion portion 109 extends and which has a substantially box shape, a universal cord 112, etc.

The insertion portion 109 of the endoscope 102 is configured, in order from the distal end side, of a distal end portion 106, a bending portion 107, and a flexible tube portion 108, which are continuously connected. A proximal end of the insertion portion 109 is connected to an operation portion 110.

Inside the distal end portion 106, one of the image pickup units (1, 1B) of the above-described embodiments is disposed.

The operation portion 110 is mainly configured of a forceps port 111 having an opening for inserting a treatment instrument, etc., an operation portion body configuring a grasping portion, a plurality of operating members provided on an outer surface of the operation portion body to perform various operations of the endoscope 102, etc.

The forceps port 111 in the operation portion 110 configures a proximal-end-side opening of the treatment instrument channel (not shown), which is inserted and arranged between the operation portion 110 and a distal-end-side opening of the distal end portion 106 of the insertion portion 109.

The universal cord 112 is a tubular member that extends from a side of the operation portion 110. A scope connector 113 is provided at a distal end part of the universal cord 112. The scope connector 113 is connected to a light source apparatus 103.

The light source apparatus 103 is an apparatus that supplies illumination light to an illumination optical member (not shown) provided inside the distal end portion 106 of the insertion portion 109 of the endoscope 102. The illumination light emitted from the light source apparatus 103 is transmitted from the scope connector 113, through a fiber-optic cable 117 that is arranged through the universal cord 112, the operation portion 110, and the insertion portion 109, to the distal end portion 106 of the insertion portion 109 of the endoscope 102. The illumination light transmits through an illumination optical member (not shown) provided on a front surface of the distal end portion 106 and is radiated toward an observation object (such as a lesion part) forward of the distal end portion 106.

A scope cable 114 extends laterally from the scope connector 113. An electrical connector portion 115 is provided at a distal end part of the scope cable 114. The electrical connector portion 115 is connected to the video processor 104.

The video processor 104 is a control apparatus that controls the entire endoscope system 100. In this case, the video processor 104 includes a signal processing circuit that receives an image pickup signal from the image pickup unit (1, 1B) provided inside the distal end portion 106 of the insertion portion 109 of the endoscope 102 and performs a predetermined signal processing, a control processing circuit that outputs a control signal, etc. to drive the image pickup unit (1, 1B), etc.

For this purpose, a signal transmission cable (not shown) is disposed between the video processor 104 and an internal component unit of the distal end portion 106 (such as, e.g. the image pickup unit (1, 1B)). The signal transmission cable is arranged, inside through, e.g., the electrical connector portion 115, the scope cable 114, the scope connector 113, the universal cord 112, the operation portion 110, and the insertion portion 109.

With this configuration, e.g., an image pickup signal output from the image pickup unit (1, 1B), a control signal output from the video processor 104, etc. are transmitted between the image pickup unit (1, 1B) and the video processor 104 through the signal transmission cable. The signal transmission cable employs, e.g., a composite cable in the form of a plurality of cables bundled together and covered with an outer shield, outer tube, etc.

A video cable 116 is used to connect between the video processor 104 and the display apparatus 105. The video cable 116 transmits an image signal, a control signal, etc. output from the video processor 104 to the display apparatus 105.

The display apparatus 105 receives the image and control signals output from the video processor 104 and displays an endoscopic image and various types of information in a predetermined form according to the received control signal.

The endoscope system 100 including the endoscope 102 that employs the image pickup unit (1, 1B) of each embodiment of the present invention is roughly configured as described above. Other configurations in the endoscope system 100 are similar to conventional endoscope systems of the same type.

The present invention is not limited to the above-described each embodiment, and it is of course possible to implement various modifications and applications within the scope that does not depart from the spirit of the invention. The above-described each embodiment includes inventions in various stages, and various inventions can be extracted by appropriate combinations of the disclosed plurality of configuration requirements. For example, even if some of the configuration requirements are deleted from all the configuration requirements shown in the above-described each embodiment, if the problem to be solved by the invention can be solved and the effect of the invention can be obtained, the configuration from which these requirements are deleted can be extracted as an invention. Furthermore, components across different embodiments may be combined as appropriate. The invention is not limited by any particular embodiment thereof except by the appended claims.

What is claimed is:

1. An image pickup unit comprising:
an image pickup device having a light receiving surface and a back surface on an opposite side to the light receiving surface;
a first substrate having a first main surface, a second main surface on an opposite side to the first main surface, and at least four side surfaces covering an area including a periphery of the first main surface and the second main surface, the first substrate comprising a stacked substrate including a plurality of internal wirings; and
a second substrate having a third main surface, a fourth main surface on an opposite side to the third main surface, and at least four side surfaces covering an area including a periphery of the third main surface and the fourth main surface, wherein
an electrode on the back surface of the image pickup device and an electrode on the first main surface of the first substrate are bonded to each other,
an electrode on the second main surface of the first substrate and an electrode on the third main surface of the second substrate are bonded to each other,
a plurality of end portions of the internal wirings are, of the four side surfaces of the first substrate, not exposed on a first side surface and a second side surface on an opposite side to the first side surface, and exposed on a third side surface and a fourth side surface orthogonal to the first side surface and the second side surface, and
the exposed end portions of the internal wirings are arranged only in a single layer of the stacked substrate of the first substrate.

2. The image pickup unit according to claim 1, wherein an electrode on the first main surface of the first substrate, an electrode on the second main surface, and an electrode on the third main surface of the second substrate comprise a plurality of electrodes coated with plating.

3. The image pickup unit according to claim 1, wherein the first and second main surfaces and the third and fourth main surfaces are parallel to each other.

4. An image pickup unit comprising:
an image pickup device having a light receiving surface and a back surface on an opposite side to the light receiving surface;
a first substrate having a first main surface, a second main surface on an opposite side to the first main surface, and at least four side surfaces covering an area including a periphery of the first main surface and the second main surface, the first substrate comprising a stacked substrate including a plurality of internal wirings; and
a second substrate having a third main surface, a fourth main surface on an opposite side to the third main surface, and at least four side surfaces covering an area including a periphery of the third main surface and the fourth main surface, wherein
an electrode on the back surface of the image pickup device and an electrode on the first main surface of the first substrate are bonded to each other,
an electrode on the second main surface of the first substrate and an electrode on the third main surface of the second substrate are bonded to each other,
a plurality of end portions of the internal wirings are, of the four side surfaces of the first substrate, not exposed on a first side surface and a second side surface on an opposite side to the first side surface, and exposed on a third side surface and a fourth side surface orthogonal to the first side surface and the second side surface, and
the plurality of exposed end portions of the internal wirings are arranged at positions that do not overlap with each other in a stacking direction.

5. The image pickup unit according to claim 1, wherein the second substrate comprises a bonding electrode to which a cable is to be bonded, on at least one of a fifth side surface parallel to the first and second side surfaces and a sixth side surface on an opposite side to the fifth side surface.

6. The image pickup unit according to claim 5, wherein
the second substrate comprises a stacked substrate including an internal wiring, and
an end portion of the internal wirings is exposed on a seventh side surface orthogonal to the fifth and sixth side surfaces, and on an eighth side surface on an opposite side to the seventh side surface.

7. The image pickup unit according to claim 6, wherein, in the second substrate, the plurality of exposed end portions of the internal wirings are arranged side by side in a direction orthogonal to a stacking direction.

8. The image pickup unit according to claim 6, wherein the plurality of exposed end portions of the internal wirings are arranged at positions that do not overlap with each other in a stacking direction.

9. The image pickup unit according to claim 6, wherein the plurality of exposed end portions of the first and second substrates are arranged side by side in a direction orthogonal to a stacking direction, and are arranged at positions that do not overlap with each other in a stacking direction.

10. An endoscope comprising an insertion portion to be inserted into a subject and an image pickup unit provided at a distal end of the insertion unit, the image pickup unit comprising:
an image pickup device having a light receiving surface and a back surface on an opposite side to the light receiving surface;
a first substrate having a first main surface, a second main surface on an opposite side to the first main surface, and at least four side surfaces covering an area including a periphery of the first main surface and the second main surface, the first substrate comprising a stacked substrate including a plurality of internal wirings; and
a second substrate having a third main surface, a fourth main surface on an opposite side to the third main surface, and at least four side surfaces covering an area including a periphery of the third main surface and the fourth main surface, wherein
an electrode on the back surface of the image pickup device and an electrode on the first main surface of the first substrate are bonded to each other,
an electrode on the second main surface of the first substrate and an electrode on the third main surface of the second substrate are bonded to each other,
a plurality of end portions of the internal wirings are, of the four side surfaces of the first substrate, not exposed on a first side surface and a second side surface on an opposite side to the first side surface, and exposed on a third side surface and a fourth side surface orthogonal to the first side surface and the second side surface, and
the exposed end portions of the internal wirings are arranged only in a single layer of the stacked substrate of the first substrate.
